(19) United States
(12) Patent Application Publication
(10) Pub. No.: US 2021/0317402 A1
(43) Pub. Date: Oct. 14, 2021

(54) GENOME-WIDE RATIONALLY-DESIGNED MUTATIONS LEADING TO ENHANCED LYSINE PRODUCTION IN E. COLI

(71) Applicant: Inscripta, Inc., Boulder, CO (US)

(72) Inventors: Eric Abbate, Boulder, CO (US); Katherine Krouse, Boulder, CO (US); Nandini Krishnamurthy, Boulder, CO (US); Richard Fox, Boulder, CO (US); Daniel Held, Boulder, CO (US); Michael Clay, Boulder, CO (US); Krishna Yerramsetty, Boulder, CO (US)

(21) Appl. No.: 17/355,295

(22) Filed: Jun. 23, 2021

Related U.S. Application Data
(63) Continuation-in-part of application No. 17/159,137, filed on Jan. 26, 2021, now Pat. No. 11,078,458.
Application No. 17/159,137 is a continuation of application No. 16/904,827, filed on Jun. 18, 2020, now Pat. No. 10,920,189.
(60) Provisional application No. 62/865,075, filed on Jun. 21, 2019.

(51) Int. Cl. C12N 1/20; C07K 14/245

(52) U.S. Cl. CPC C12N 1/20; C12N 2511/00; C12N 2510/02; C07K 14/245

(57) ABSTRACT

The present disclosure relates to various different types of variants in E. coli coding and noncoding regions leading to enhanced lysine production for, e.g., supplements and nutraceuticals.

### RELATED APPLICATIONS

This application is a continuation-in-part of U.S. Ser. No. 17/159,137, filed 26 Jan. 2021, entitled “Genome-Wide Rationally-Designed Mutations Leading to Enhanced Lysine Production in E. coli”; which is a continuation of U.S. Ser. No. 16/904,827, filed 18 Jun. 2020, entitled “Genome-Wide Rationally-Designed Mutations Leading to Enhanced Lysine Production in E. coli”, now U.S. Pat. No. 10,920,189; both of which claim priority to U.S. Provisional Applications Nos: 62/865,075, filed 21 Jun. 2019, entitled “Genome-Wide Rationally-Designed Mutations Leading to Enhanced Lysine Production in E. coli”, incorporated by reference herein in its entirety.

### INCORPORATION BY REFERENCE

Submitted with the present application is an electronically filed sequence listing via EFS-Web as an ASCII formatted sequence listing, entitled “INSC046CIP_seqlist_20210622”, created Jun. 22, 2021, and 1,565,000 bytes in size. The sequence listing is part of the specification filed herewith and is incorporated by reference in its entirety.

### FIELD OF THE INVENTION

The present disclosure relates to mutations in genes in E. coli leading to enhanced lysine production.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

The amino acid lysine is an α-amino acid that is used in the biosynthesis of proteins and is a metabolite of E. coli, S. cerevisiae, plants, humans and other mammals, as well as algae. Lysine contains an α-amino group, an α-carboxylic acid group, and has a chemical formula of C₆H₁₄N₂O₂ One of nine essential amino acids in humans, lysine is required for growth and tissue repair and has a role as a micronutrient, a nutraceutical, an agricultural feed supplement, an anticonvulsant, as well as a precursor for the production of peptides. Because of these roles as, e.g., a supplement and nutraceutical, there has been a growing effort to produce lysine on a large scale.

Accordingly, there is a need in the art for organisms that produce enhanced amounts of lysine where such organisms can be harnessed for large scale lysine production. The disclosed nucleic acid sequences from E. coli satisfy this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure provides variant E. coli genes and non-coding sequences that produce enhanced amounts of lysine in culture including double and triple combinations of variant sequences. Thus, in some embodiments, the present disclosure provides any one of E. coli variants comprising the sequences in SEQ ID NOs: 2-324 and combinations thereof.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIGS. 1A and 1B are graphic depictions of the lysine pathway in E. coli, highlighting the enzymes in the pathway targeted for rationally-designed editing. FIG. 1B is a continuation of FIG. 1A.

FIG. 2 enumerates the biological target, edit outcome, edit type and scale for the initial 200,000 edits made to the E. coli lysine pathway.

FIG. 3A is an exemplary engine vector for creating edits in E. coli. FIG. 3B is an exemplary editing vector for creating edits in E. coli.

It should be understood that the drawings are not necessarily to scale, and that like reference numbers refer to like features.

### DETAILED DESCRIPTION

All of the functionalities described in connection with one embodiment of the methods, devices or instruments described herein are intended to be applicable to the additional embodiments of the methods, devices and instruments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions molecular biology (including recombinant techniques), cell biology, biochemistry, and genetic engineering technology, which are within the skill of those who practice in the art. Such conventional techniques and descriptions can be found in standard laboratory manuals such as Green and Sambrook, Molecular Cloning: A Laboratory Manual. 4th, ed., Cold Spring Harbor Laboratory Press, Cold Spring Harbor, N.Y., (2014); Current Protocols in Molecular Biology, Ausubel, et al. eds., (2017); Neumann, et al., Electroporation and Electrofusion in Cell Biology, Plenum Press, New York, 1989; and Chang, et al., Guide to Electroporation and Electrofusion, Academic Press, California (1992), all of which are herein incorporated in their entirety by reference for all purposes.

Note that as used herein and in the appended claims, the singular forms “a,” “an,” and “the” include plural referents unless the context clearly dictates otherwise. Thus, for example, reference to “a cell” refers to one or more cells, and reference to “the system” includes reference to equivalent steps, methods and devices known to those skilled in the art, and so forth.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and-for some components-translated in an appropriate host cell.

The term “CREATE cassette” or “editing cassette” refers to a gRNA linked to a donor DNA or HA. Methods and compositions for designing and synthesizing CREATE editing cassettes are described in U.S. Pat. Nos. 10,240,167; 10,266,849; 9,982,278; 10,351,877; 10,364,442; 10,435,715; and 10,465,207; and U.S. Ser. No. 16/550,092, filed 23 Aug. 2019; Ser. No. 16/551,517, filed 26 Aug. 2019; Ser. No. 16/773,618, filed 27 Jan. 2020; and Ser. No. 16/773,712, filed 27 Jan. 2020, all of which are incorporated by reference herein in their entirety.

As used herein the term “donor DNA” or “donor nucleic acid” refers to nucleic acid that is designed to introduce a DNA sequence modification (insertion, deletion, substitution) into a locus (e.g., a target genomic DNA sequence or cellular target sequence) by homologous recombination using nucleic acid-guided nucleases. For homology-directed repair, the donor DNA must have sufficient homology to the regions flanking the “cut site” or site to be edited in the genomic target sequence. The length of the homology arm(s) will depend on, e.g., the type and size of the modification being made. In many instances and preferably, the donor DNA will have two regions of sequence homology (e.g., two homology arms) to the genomic target locus. Preferably, an “insert” region or “DNA sequence modification” region—the nucleic acid modification that one desires to be introduced into a genome target locus in a cell—will be located between two regions of homology. The DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of the genomic target sequence. A deletion or insertion may be a deletion or insertion of 1, 2, 3, 4, 5, 10, 15, 20, 25, 30, 40, 50, 75, 100, 150, 200, 300, 400, or 500 or more base pairs of the genomic target sequence.

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease.

“Homology” or “identity” or “similarity” refers to sequence similarity between two peptides or, more often in the context of the present disclosure, between two nucleic acid molecules. The term “homologous region” or “homology arm” refers to a region on the donor DNA with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

As used herein, the terms “protein” and “polypeptide” are used interchangeably. Proteins may or may not be made up entirely of amino acids.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA transcribed by any class of any RNA polymerase I, II or III. Promoters may be constitutive or inducible, and in some embodiments the transcription of at least one component of the nucleic acid-guided nuclease editing system is—and often at least three components of the nucleic acid-guided nuclease editing system are—under the control of an inducible promoter. A number of gene regulation control systems have been developed for the controlled expression of genes in plant, microbe, and animal cells, including mammalian cells, including the pL promoter (induced by heat inactivation of the CI857 repressor), the pPhIF promoter (induced by the addition of 2,4 diacetylphloroglucinol (DAPG)), the pBAD promoter (induced by the addition of arabinose to the cell growth medium), and the rhamnose inducible promoter (induced by the addition of rhamnose to the cell growth medium). Other systems include the tetracycline-controlled transcriptional activation system (Tet-On/Tet-Off, Clontech, Inc. (Palo Alto, Calif.); Bujard and Gossen, PNAS, 89(12): 5547-5551 (1992)), the Lac Switch Inducible system (Wyborski et al., Environ Mol Mutagen, 28(4): 447-58 (1996); DuCoeur et al., Strategies 5(3): 70-72 (1992); U.S. Pat. No. 4,833,080), the ecdysone-inducible gene expression system (No et al., PNAS, 93(8): 3346-3351 (1996)), the cumate gene-switch system (Mullick et al., BMC Biotechnology, 6: 43 (2006)), and the tamoxifen-inducible gene expression (Zhang et al., Nucleic Acids Research, 24: 543-548 (1996)) as well as others.

As used herein the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, nourseothricin N-acetyl transferase, chloramphenicol, erythromycin, tetracycline, gentamicin, bleomycin, streptomycin, rifampicin, puromycin, hygromycin, blasticidin, and G418 may be employed. In other embodiments, selectable markers include, but are not limited to sugars such as rhamnose. “Selective medium” as used herein refers to cell growth medium to which has been added a chemical compound or biological moiety that selects for or against selectable markers.

The term “specifically binds” as used herein includes an interaction between two molecules, e.g., an engineered peptide antigen and a binding target, with a binding affinity represented by a dissociation constant of about 10⁻⁷ M, about 10⁻⁸ M, about 10⁻⁹ M, about 10⁻¹⁰ M, about 10⁻¹¹M, about 10⁻¹²M, about 10⁻¹³M, about 10⁻¹⁴M or about 10⁻¹⁵M.

The terms “target genomic DNA sequence”, “cellular target sequence”, or “genomic target locus” refer to any locus in vitro or in vivo, or in a nucleic acid (e.g., genome) of a cell or population of cells, in which a change of at least one nucleotide is desired using a nucleic acid-guided nuclease editing system. The cellular target sequence can be a genomic locus or extrachromosomal locus.

The term “variant” may refer to a polypeptide or polynucleotide that differs from a reference polypeptide or polynucleotide but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be a conservatively modified variant. A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, synthetic chromosomes, and the like. As used herein, the phrase “engine vector” comprises a coding sequence for a nuclease to be used in the nucleic acid-guided nuclease systems and methods of the present disclosure. The engine vector also comprises in E. coli, the λ Red recombineering system or an equivalent thereto which repairs the double-stranded breaks resulting from the cut by the nuclease. Engine vectors also typically comprise a selectable marker. As used herein the phrase “editing vector” comprises a donor nucleic acid, optionally including an alteration to the cellular target sequence that prevents nuclease binding at a PAM or spacer in the cellular target sequence after editing has taken place, and a coding sequence for a gRNA. The editing vector may also and preferably does comprise a selectable marker and/or a barcode. In some embodiments, the engine vector and editing vector may be combined; that is, all editing and selection components may be found on a single vector. Further, the engine and editing vectors comprise control sequences operably linked to, e.g., the nuclease coding sequence, recombineering system coding sequences (if present), donor nucleic acid, guide nucleic acid(s), and selectable marker(s).

### Library Design Strategy and Nuclease-Directed Genome Editing

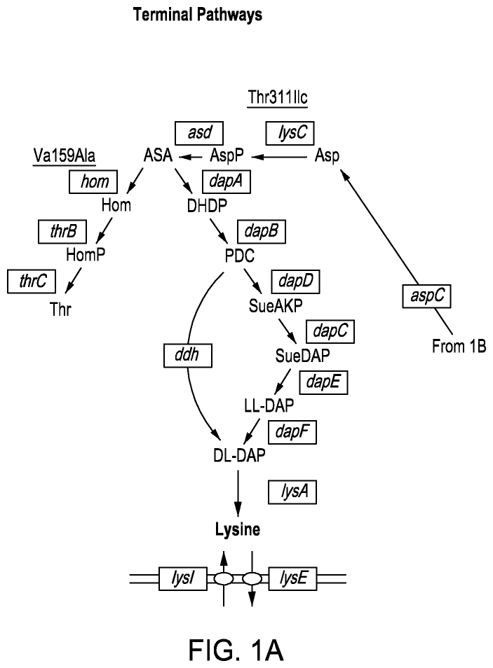

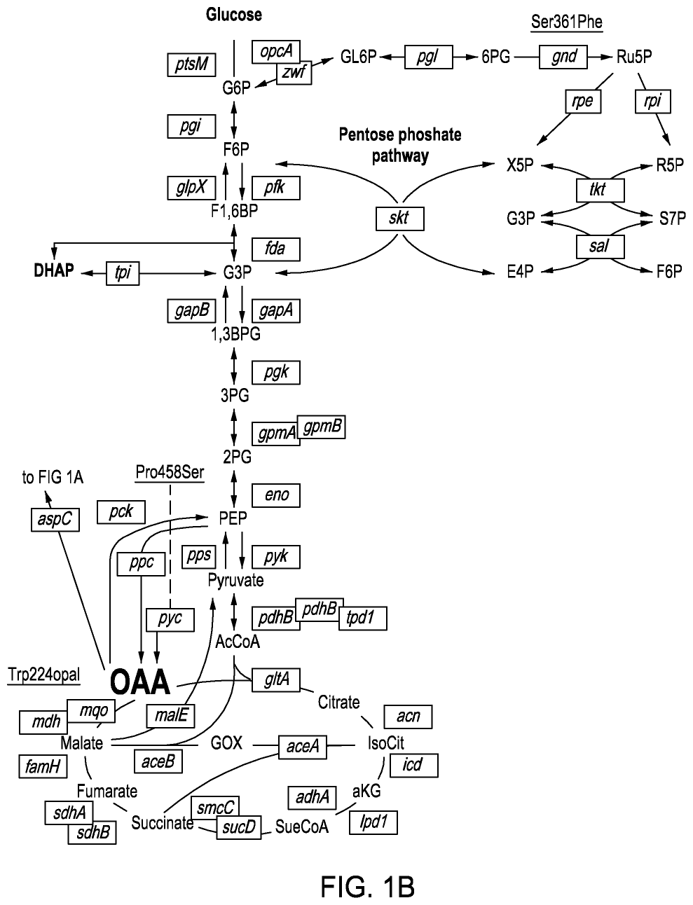

Lysine is naturally synthesized in E. coli along the diaminopimelate (DAP) biosynthetic pathway. See, e.g., FIG. 1. Strain engineering strategies for increasing lysine production in E. coli and other industrially-relevant production hosts such as Corynebacterium glutamicum have historically focused on the genes in the DAP pathway as obvious targets for mutagenesis and over-expression. Beyond this short list of genes encoding the lysine biosynthetic enzymes, it is likely that additional loci throughout the E. coli genome may also contribute appreciably (if less directly) to improved lysine yields in an industrial production setting. For this reason, targeted mutagenesis strategies which enable a broader query of the entire genome are also of significant value to the lysine metabolic engineer.

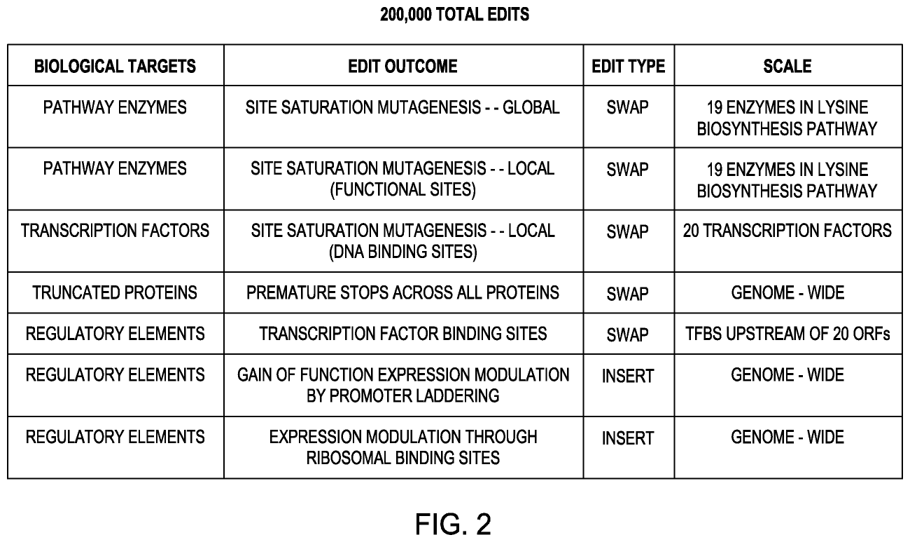

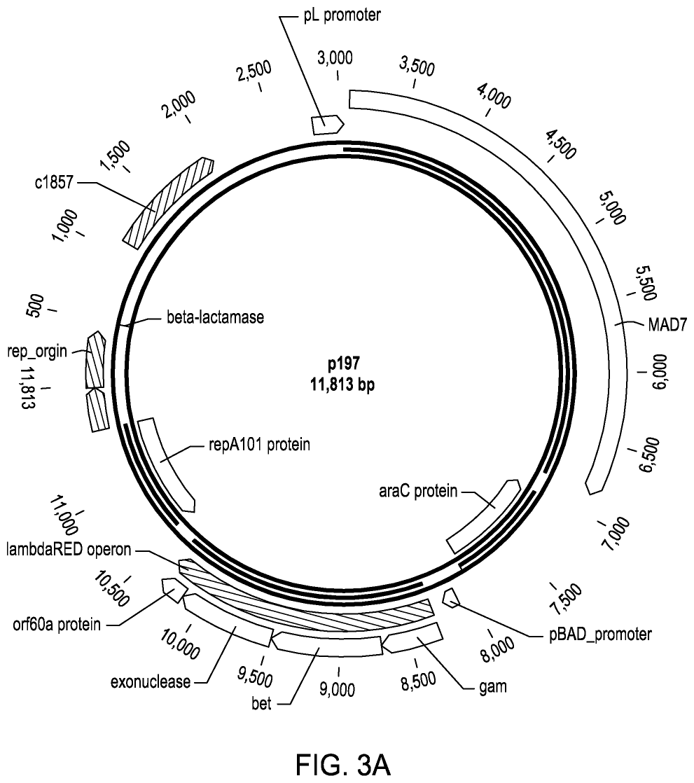

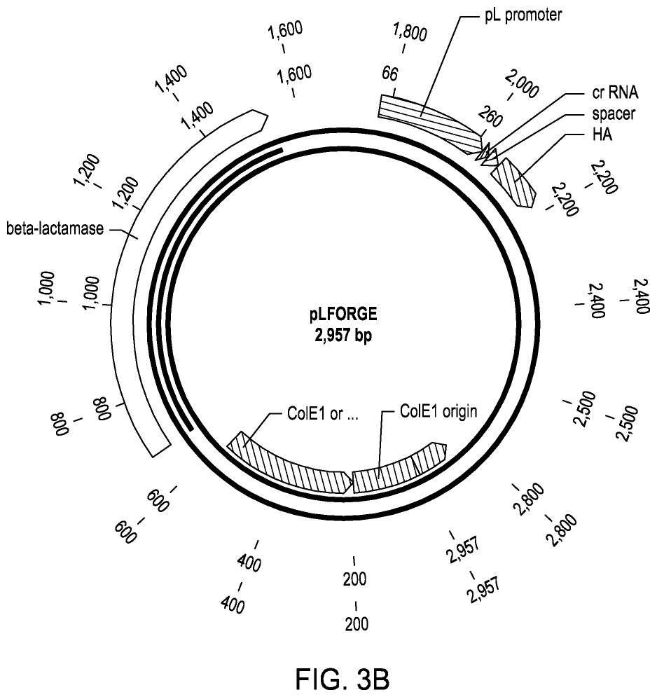

The variants presented in this disclosure are the result of nucleic acid-guided nuclease editing of 200,000 unique and precise designs at specified loci around the genome in a wildtype strain of E. coli harboring an engine plasmid such as that shown in FIG. 3A (such transformed MG1655 strain is referred to herein as E. coli strain EC83) and using the resulting lysine production levels to conduct additional nucleic acid-guided nuclease editing in two engineered strains of MG1655 to produce double- and triple-variant engineered strains. The first engineered strain is strain MG1655 with a single mutation comprising dapA E84T (SEQ ID NO: 1), the lysine production for which was approximately 500-fold over wildtype lysine production in MG1655. The second engineered strain is strain MG1655 with a double mutation comprising dapA E84T (SEQ ID NO: 1) and dapA J23100 (a mutation in the E. coli dapA promoter, SEQ ID NO. 2), the lysine production for which was approximately 10,000-fold over wildtype lysine production. See, e.g., FIG. 2 for a summary of the types of edits included in the 200,000 editing vectors used to generate the variants. The engine plasmid comprises a coding sequence for the MAD7 nuclease under the control of the inducible pL promoter, the λ Red operon recombineering system under the control of the inducible pBAD promoter (inducible by the addition of arabinose in the cell growth medium), the c1857 gene under the control of a constitutive promoter, as well as a selection marker and an origin of replication. As described above, the λ Red recombineering system repairs the double-stranded breaks resulting from the cut by the MAD7 nuclease. The c1857 gene at 30° C. actively represses the pL promoter (which drives the expression of the MAD7 nuclease and the editing or CREATE cassette on the editing cassette such as the exemplary editing vector shown in FIG. 3B); however, at 42° C., the c1857 repressor gene unfolds or degrades, and in this state the c1857 repressor protein can no longer repress the pL promoter leading to active transcription of the coding sequence for the MAD7 nuclease and the editing (e.g., CREATE) cassette.

FIG. 3B depicts an exemplary editing plasmid comprising the editing (e.g, CREATE) cassette (crRNA, spacer and HA) driven by a pL promoter, a selection marker, and an origin of replication.

Mutagenesis libraries specifically targeting the genes in the DAP pathway—along with a number of genes whose enzymes convert products feeding into the DAP pathway—were designed for saturation mutagenesis. Additionally, to more deeply explore the rest of the genome for new targets involved in lysine biosynthesis, libraries were designed to target all annotated loci with either premature stop codons (for a knock-out phenotype) or insertion of a set of five synthetic promoter variants (for expression modulation phenotypes).

The 200,000 nucleic acid mutations or edits described herein were generated using MAD7, along with a gRNA and donor DNA. A nucleic acid-guided nuclease such as MAD7 is complexed with an appropriate synthetic guide nucleic acid in a cell and can cut the genome of the cell at a desired location. The guide nucleic acid helps the nucleic acid-guided nuclease recognize and cut the DNA at a specific target sequence. By manipulating the nucleotide sequence of the guide nucleic acid, the nucleic acid-guided nuclease may be programmed to target any DNA sequence for cleavage as long as an appropriate protospacer adjacent motif (PAM) is nearby. In certain aspects, the nucleic acid-guided nuclease editing system may use two separate guide nucleic acid molecules that combine to function as a guide nucleic acid, e.g., a CRISPR RNA (crRNA) and trans-activating CRISPR RNA (tracrRNA). In other aspects, the guide nucleic acid may be a single guide nucleic acid that includes both the crRNA and tracrRNA sequences.

Again, the resulting lysine production levels from the single variants were used to conduct additional nucleic acid-guided nuclease editing in two engineered strains of MG1655 to produce double- and triple-variant engineered strains. The first engineered strain is strain MG1655 with a single mutation comprising dapA E84T (SEQ ID NO: 1), the lysine production for which was approximately 500-fold over wildtype lysine production in MG1655. The second engineered strain is strain MG1655 with a double mutation comprising dapA E84T (SEQ ID NO: 1) and dapA J23100 (a mutation in the E. coli dapA promoter, SEQ ID NO. 2), the lysine production for which was approximately 10,000-fold over wildtype lysine production.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In the methods to generate the 200,000 member library, the guide nucleic acids were provided as a sequence to be expressed from a plasmid or vector comprising both the guide sequence and the scaffold sequence as a single transcript under the control of an inducible promoter. The guide nucleic acids are engineered to target a desired target sequence by altering the guide sequence so that the guide sequence is complementary to a desired target sequence, thereby allowing hybridization between the guide sequence and the target sequence. In general, to generate an edit in the target sequence, the gRNA/nuclease complex binds to a target sequence as determined by the guide RNA, and the nuclease recognizes a protospacer adjacent motif (PAM) sequence adjacent to the target sequence. The target sequences for the genome-wide mutagenesis here encompassed 200,000 unique and precise designs at specified loci around the genome throughout the E. coli genome.

The guide nucleic acid may be and in the processes generating the variants reported herein were part of an editing cassette that also encoded the donor nucleic acid. The target sequences are associated with a proto-spacer mutation (PAM), which is a short nucleotide sequence recognized by the gRNA/nuclease complex. The precise preferred PAM sequence and length requirements for different nucleic acid-guided nucleases vary; however, PAMs typically are 2-7 base-pair sequences adjacent or in proximity to the target sequence and, depending on the nuclease, can be 5′ or 3′ to the target sequence.

In certain embodiments, the genome editing of a cellular target sequence both introduces the desired DNA change to the cellular target sequence and removes, mutates, or renders inactive a proto-spacer mutation (PAM) region in the cellular target sequence. Rendering the PAM at the cellular target sequence inactive precludes additional editing of the cell genome at that cellular target sequence, e.g., upon subsequent exposure to a nucleic acid-guided nuclease complexed with a synthetic guide nucleic acid in later rounds of editing. Thus, cells having the desired cellular target sequence edit and an altered PAM can be selected for by using a nucleic acid-guided nuclease complexed with a synthetic guide nucleic acid complementary to the cellular target sequence. Cells that did not undergo the first editing event will be cut rendering a double-stranded DNA break, and thus will not continue to be viable. The cells containing the desired cellular target sequence edit and PAM alteration will not be cut, as these edited cells no longer contain the necessary PAM site and will continue to grow and propagate.

As for the nuclease component of the nucleic acid-guided nuclease editing system, a polynucleotide sequence encoding the nucleic acid-guided nuclease can be codon optimized for expression in particular cell types, such as archaeal, prokaryotic or eukaryotic cells. The choice of nucleic acid-guided nuclease to be employed depends on many factors, such as what type of edit is to be made in the target sequence and whether an appropriate PAM is located close to the desired target sequence. Nucleases of use in the methods described herein include but are not limited to Cas 9, Cas 12/CpfI, MAD2, or MAD7 or other MADzymes. As with the guide nucleic acid, the nuclease is encoded by a DNA sequence on a vector (e.g., the engine vector—see FIG. 3A) and be under the control of an inducible promoter. In some embodiments—such as in the methods described herein—the inducible promoter may be separate from but the same as the inducible promoter controlling transcription of the guide nucleic acid; that is, a separate inducible promoter drives the transcription of the nuclease and guide nucleic acid sequences but the two inducible promoters may be the same type of inducible promoter (e.g., both are pL promoters). Alternatively, the inducible promoter controlling expression of the nuclease may be different from the inducible promoter controlling transcription of the guide nucleic acid; that is, e.g., the nuclease may be under the control of the pBAD inducible promoter, and the guide nucleic acid may be under the control of the pL inducible promoter.

Another component of the nucleic acid-guided nuclease system is the donor nucleic acid comprising homology to the cellular target sequence. In some embodiments, the donor nucleic acid is on the same polynucleotide (e.g., editing vector or editing cassette) as the guide nucleic acid. The donor nucleic acid is designed to serve as a template for homologous recombination with a cellular target sequence nicked or cleaved by the nucleic acid-guided nuclease as a part of the gRNA/nuclease complex. A donor nucleic acid polynucleotide may be of any suitable length, such as about or more than about 20, 25, 50, 75, 100, 150, 200, 500, or 1000 nucleotides in length. In certain preferred aspects, the donor nucleic acid can be provided as an oligonucleotide of between 20-300 nucleotides, more preferably between 50-250 nucleotides. The donor nucleic acid comprises a region that is complementary to a portion of the cellular target sequence (e.g., a homology arm). When optimally aligned, the donor nucleic acid overlaps with (is complementary to) the cellular target sequence by, e.g., about 20, 25, 30, 35, 40, 50, 60, 70, 80, 90 or more nucleotides. In many embodiments, the donor nucleic acid comprises two homology arms (regions complementary to the cellular target sequence) flanking the mutation or difference between the donor nucleic acid and the cellular target sequence. The donor nucleic acid comprises at least one mutation or alteration compared to the cellular target sequence, such as an insertion, deletion, modification, or any combination thereof compared to the cellular target sequence. Various types of edits were introduced herein, including site-directed mutagenesis, saturation mutagenesis, promoter swaps and ladders, knock-in and knock-out edits, SNP or short tandem repeat swaps, and start/stop codon exchanges.

In addition to the donor nucleic acid, an editing cassette may comprise one or more primer sites. The primer sites can be used to amplify the editing cassette by using oligonucleotide primers; for example, if the primer sites flank one or more of the other components of the editing cassette. In addition, the editing cassette may comprise a barcode. A barcode is a unique DNA sequence that corresponds to the donor DNA sequence such that the barcode can identify the edit made to the corresponding cellular target sequence. The barcode typically comprises four or more nucleotides. In some embodiments, the editing cassettes comprise a collection or library gRNAs and of donor nucleic acids representing, e.g., gene-wide or genome-wide libraries of gRNAs and donor nucleic acids. The library of editing cassettes is cloned into vector backbones where, e.g., each different donor nucleic acid is associated with a different barcode.

Variants of interest include those listed in Table 1 below:

TABLE 1
Variants

| | Phenotype | Phenotype |
|---|---|---|
| Mutant | FOWT | FIOPC |
| Single edit: dapA E84T | 500 | 0 |
| Single edit: dapA J21300 | 1000 | 2 |
| Triple edit: dapA E84T; dapA J21300; lysC V339P | 13.500 | 27 |
| Triple edit: dapA E84T; dapA; garD J23101 | 13.000 | 26 |
| Triple edit: dapA E84T; dapA; yicL J23100 | 13.400 | 26.8 |
| Triple edit: dapA E84T; dapA; lysP R15*** | 14.600 | 29.2 |
| Triple edit: dapA E84T; dapA; mgsA J23100 | 13.300 | 26.6 |
| Triple edit: dapA E84T; dapA; pckE100Q | 13.400 | 26.8 |
| Double edit: dapA J21300; amyA J23100 | 804.620 | 1.609 |
| Double edit: dapA J21300; amyA P15*** | 784.779 | 1.570 |
| Double edit: dapA J21300; cysN L5*** | 1320.758 | 2.642 |
| Double edit: dapA J21300; dosP J23100 | 1067.701 | 2.135 |
| Double edit: dapA J21300; emrE J23100 | 1016.806 | 2.034 |
| Double edit: dapA J21300; focB J23100 | 913.339 | 1.827 |
| Double edit: dapA J21300; glnD J23100 | 1397.503 | 2.795 |
| Double edit: dapA J21300; glnE V15*** | 1085.446 | 2.171 |
| Double edit: dapA J21300; hicB J23100 | 758.057 | 1.516 |
| Double edit: dapA J21300; maeB J23100 | 946.484 | 1.893 |
| Double edit: dapA J21300; marA Y107D | 798.469 | 1.597 |
| Double edit: dapA J21300; metL R241E | 726.648 | 1.453 |
| Double edit: dapA J21300; mfd Y5*** | 983.267 | 1.967 |
| Double edit: dapA J21300; nupX R5*** | 884.027 | 1.768 |
| Double edit: dapA J21300; pck H232G | 1409.458 | 2.819 |
| Double edit: dapA J21300; phoB J23100 | 781.383 | 1.563 |
| Double edit: dapA J21300; purM J23100 | 1633.414 | 3.267 |
| Double edit: dapA J21300; rlmL F5*** | 834.477 | 1.669 |
| Double edit: dapA J21300; wzxB K5*** | 793.985 | 1.588 |
| Double edit: dapA J21300; ydgl J23100 | 1554.101 | 3.108 |
| Double edit: dapA J21300; ydjE J23100 | 778.514 | 1.557 |
| Double edit: dapA J21300; yicL J23100 | 854.283 | 1.709 |
| Double edit: dapA J21300; yliE J23100 | 979.740 | 1.959 |
| Double edit: dapA J21300; yohF J23100 | 858.181 | 1.716 |
| Double edit: dapA J21300; ytfP N15*** | 781.981 | 1.564 |
| Double edit: dapA J21300; marA R94* | 728.433 | 1.457 |
| Double edit: dapA J21300; marA Y107K | 733.943 | 1.468 |
| Double edit: dapA J21300; metL P240D | 726.648 | 1.453 |
| Double edit: dapA J21300; metL V235C | 708.124 | 1.416 |
| Double edit: dapA J21300; pck G64D | 718.020 | 1.436 |
| Double edit: dapA J21300; setB J23100 | 727.174 | 1.454 |
| Double edit: dapA J21300; ydfO J23100 | 701.255 | 1.403 |
| Double edit: dapA J21300; ydgD J23100 | 716.198 | 1.432 |
| Double edit: dapA J21300; yejG J23100 | 731.562 | 1.463 |
| Triple edit: dapA E84T; dapA J23100; metL R241E | #N/A | #N/A |
| Triple edit: dapA E84T; dapA J23100; glnE V15*** | #N/A | #N/A |
| Triple edit: dapA E84T; dapA J23100; glnD J23100 | #N/A | #N/A |
| Triple edit: dapA E84T; dapA J23100; ytfP N15*** | #N/A | #N/A |
| Quadruple edit: dapA E84T; dapA J23100; | #N/A | #N/A |
| purM J23100; dosP J23100 | | |
| Quadruple edit: dapA E84T; dapA J23100; | #N/A | #N/A |
| purM J23100; glnD J23100 | | |
| Quadruple edit: dapA E84T; dapA J23100; | #N/A | #N/A |
| pck A532S; glnD J23100 | | |
| Triple edit: dapA E84T; pck A532S; metL R241E | #N/A | #N/A |
| Triple edit: dapA E84T; pck A532S; lysC V339P | #N/A | #N/A |
| Triple edit: dapA E84T; pck A532S; glnE V15*** | #N/A | #N/A |
| Triple edit: dapA E84T; pck A532S; glnD J23100 | #N/A | #N/A |
| Double edit: dapA E84T; azoR J23100 | #N/A | #N/A |
| Double edit: dapA E84T; cra ESA | #N/A | #N/A |
| Double edit: dapA E84T; cra I6S | #N/A | #N/A |
| Double edit: dapA E84T; cra S18L | #N/A | #N/A |
| Double edit: dapA E84T; dtpA J23100 | #N/A | #N/A |
| Double edit: dapA E84T; fnr R213A | #N/A | #N/A |
| Double edit: dapA E84T; ftsY J23100 | #N/A | #N/A |
| Double edit: dapA E84T; glnD J23100 | #N/A | #N/A |
| Double edit: dapA E84T; gpp J23100 | #N/A | #N/A |
| Double edit: dapA E84T; lysC V339N | #N/A | #N/A |
| Double edit: dapA E84T; lysC V339P | #N/A | #N/A |
| Double edit: dapA E84T; marA K106D | #N/A | #N/A |
| Double edit: dapA E84T; marA Y107M | #N/A | #N/A |
| Double edit: dapA E84T; marA K106D | #N/A | #N/A |
| Double edit: dapA E84T; marA M109Q | #N/A | #N/A |
| Double edit: dapA E84T; metL A50N | #N/A | #N/A |
| Double edit: dapA E84T; metL R207C | #N/A | #N/A |
| Double edit: dapA E84T; metL S230V | #N/A | #N/A |
| Double edit: dapA E84T; metL W2291I | #N/A | #N/A |
| Double edit: dapA E84T; mgsA JS23100 | #N/A | #N/A |
| Double edit: dapA E84T; moaC M15*** | #N/A | #N/A |
| Double edit: dapA E84T; oppD Q15*** | #N/A | #N/A |
| Double edit: dapA E84T; rob K93P | #N/A | #N/A |
| Double edit: dapA E84T; ycgI J23100 | #N/A | #N/A |
| Double edit: dapA E84T; yeeP J23100 | #N/A | #N/A |
| Double edit: dapA E84T; yihN J23100 | #N/A | #N/A |
| Double edit: dapA E84T; yohO K5*** | #N/A | #N/A |
| Single edit: metF S15*** | #N/A | #N/A |
| Single edit: glgB A15*** | #N/A | #N/A |
| Single edit: yibL E5*** | #N/A | #N/A |
| Single edit: ppc R880W | #N/A | #N/A |
| Single edit: metE H641N | #N/A | #N/A |
| Single edit: metE C726H | #N/A | #N/A |
| Single edit: rffG V216D | #N/A | #N/A |
| Single edit: scpB G65N | #N/A | #N/A |
| Single edit: lysA H373Y | #N/A | #N/A |
| Single edit: lysA N58I | #N/A | #N/A |
| Single edit: lysC L377R | #N/A | #N/A |
| Single edit: tktB E1915 | #N/A | #N/A |
| Single edit: tktB K342S | #N/A | #N/A |
| Single edit: tktB Q341K | #N/A | #N/A |
| Single edit: lysC T343I | #N/A | #N/A |
| Single edit: tktB F283F | #N/A | #N/A |
| Single edit: lysA M2318K | #N/A | #N/A |
| Single edit: tktB I102W | #N/A | #N/A |
| Single edit: dapA G76Y | #N/A | #N/A |
| Single edit: pck E239Y | #N/A | #N/A |
| Single edit: pck G208Q | #N/A | #N/A |
| Single edit: pck K106W | #N/A | #N/A |
| Single edit: pck S234C | #N/A | #N/A |
| Single edit: pck T176E | #N/A | #N/A |
| Single edit: pck N177C | #N/A | #N/A |
| Single edit: pck T499Y | #N/A | #N/A |
| Single edit: pck V55Q | #N/A | #N/A |
| Single edit: dapA L88A | #N/A | #N/A |
| Single edit: pck V48C | #N/A | #N/A |
| Single edit: pck V371H | #N/A | #N/A |
| Single edit: selA I15*** | #N/A | #N/A |
| Single edit: xylH T15*** | #N/A | #N/A |
| Single edit: allC W15*** | #N/A | #N/A |
| Single edit: metB Q5*** | #N/A | #N/A |
| Single edit: metB N15*** | #N/A | #N/A |
| Single edit: metL J23100 | #N/A | #N/A |
| Single edit: nirB J23100 | #N/A | #N/A |
| Single edit: yrhC J23100 | #N/A | #N/A |
| Single edit: prlC J23100 | #N/A | #N/A |
| Single edit: argK J23100 | #N/A | #N/A |
| Single edit: glgP J23100 | #N/A | #N/A |
| Single edit: argA J23100 | #N/A | #N/A |
| Single edit: tmpR J23112 | #N/A | #N/A |
| Single edit: ugpQ J23112 | #N/A | #N/A |
| Single edit: bscF J23112 | #N/A | #N/A |
| Single edit: lafU J23112 | #N/A | #N/A |
| Single edit: prfH J23112 | #N/A | #N/A |
| Single edit: csiR J23106 | #N/A | #N/A |
| Single edit: feoA J23106 | #N/A | #N/A |
| Single edit: glgP J23106 | #N/A | #N/A |
| Single edit: dapA H56M | #N/A | #N/A |
| Single edit: dapA A79E | #N/A | #N/A |
| Single edit: dapA H56K | #N/A | #N/A |
| Single edit: dapA N80H | #N/A | #N/A |
| Single edit: dapA A79H | #N/A | #N/A |
| Single edit: dapA A79V | #N/A | #N/A |
| Single edit: dapA H56T | #N/A | #N/A |
| Single edit: dapA A79L | #N/A | #N/A |
| Single edit: dapF L45E | #N/A | #N/A |
| Single edit: pck M231L | #N/A | #N/A |
| Single edit: pck G107V | #N/A | #N/A |
| Single edit: dapA H56E | #N/A | #N/A |
| Single edit: dapA N80Q | #N/A | #N/A |
| Single edit: dapA A79N | #N/A | #N/A |
| Single edit: pck H232L | #N/A | #N/A |
| Single edit: dapA A79Y | #N/A | #N/A |
| Single edit: dapA H56Y | #N/A | #N/A |
| Single edit: dapA L88S | #N/A | #N/A |
| Single edit: dapA E84N | #N/A | #N/A |
| Single edit: lysA G148A | #N/A | #N/A |
| Single edit: lysP R15*** | #N/A | #N/A |
| Single edit: dapF N190I | #N/A | #N/A |
| Double edit: dapA A79H; dapA D65S | #N/A | #N/A |
| Single edit: lysC V339A | #N/A | #N/A |
| Single edit: yfcO J23112 | #N/A | #N/A |
| Single edit: aroE K15*** | #N/A | #N/A |
| Single edit: metL A772R | #N/A | #N/A |
| Single edit: metL D508H | #N/A | #N/A |
| Single edit: yijF J23101 | #N/A | #N/A |
| Single edit: dapA A81Q | #N/A | #N/A |
| Single edit: garD Q274D | #N/A | #N/A |
| Single edit: dapA L88D | #N/A | #N/A |
| Single edit: garD J23101 | #N/A | #N/A |
| Single edit: lysC V339D | #N/A | #N/A |
| Single edit: dapF L46N | #N/A | #N/A |
| Single edit: ribE J23101 | #N/A | #N/A |
| Single edit: lysC S338K | #N/A | #N/A |
| Single edit: argE L5*** | #N/A | #N/A |
| Single edit: acrD J23106 | #N/A | #N/A |
| Single edit: dapA L88F | #N/A | #N/A |
| Single edit: dapA Y106H | #N/A | #N/A |
| Single edit: yidR V5*** | #N/A | #N/A |
| Single edit: yicL R5*** | #N/A | #N/A |
| Single edit: dapA N80A | #N/A | #N/A |
| Single edit: hupA Q5*** | #N/A | #N/A |
| Single edit: yhjR G15*** | #N/A | #N/A |
| Single edit: yhdJ C5*** | #N/A | #N/A |
| Single edit: adeQ T15*** | #N/A | #N/A |
| Single edit: dppF A15*** | #N/A | #N/A |
| Single edit: dapA A81V | #N/A | #N/A |
| Single edit: dapA A57M | #N/A | #N/A |
| Single edit: yidG D3*** | #N/A | #N/A |
| Single edit: pck K106I | #N/A | #N/A |
| Single edit: metL P766F | #N/A | #N/A |
| Triple edit: dapA E84T; dapA J23100; thrA G502P | ctrl not run | 18.99929877 |
| Double edit: dapA E84T; thrA K682G | ctrl not run | 13.97383116 |
| Double edit: dapA E84T; thrA G634I | ctrl not run | 17.45976727 |
| Double edit: dapA E84T; thrA Q487D | ctrl not run | 11.08323335 |
| Double edit: dapA E84T; thrA A808L | ctrl not run | 6.250993725 |
| Double edit: dapA E84T; thrA G502P | ctrl not run | 3.112729503 |
| Single edit: thrA V609N | 745.1695991 | 2.771991579 |
| Single edit: thrA G477E | 779.8274067 | 2.900916794 |
| Single edit: thrA F603K | 514.3469754 | 1.913343601 |
| Single edit: thrA L480R | 387.0459064 | 1.439790344 |
| Single edit: thrA G474L | 422.3582282 | 1.571150317 |
| Single edit: thrA I685W | 721.3030528 | 2.683209286 |
| Single edit: thrA S637W | 70.21122624 | 0.261182056 |
| Single edit: thrA G626E | 1.553737174 | 0.00577982 |
| Single edit: thrA A777* | 1815.768654 | 3.485792728 |
| Single edit: thrA G626* | 1237.618292 | 2.37589785 |
| Single edit: thrA A581* | 1029.134432 | 1.975664306 |
| Single edit: thrA L683* | 964.9281563 | 1.85240534 |
| Single edit: thrA M677* | 934.2252013 | 1.793463835 |
| Single edit: thrA R671* | 837.8471306 | 1.60844358 |
| Single edit: thrA M586* | 732.9563684 | 1.407081223 |
| Single edit: thrA R744* | 553.2479145 | 1.062088803 |
| Single edit: thrA G638* | 550.9389894 | 1.057656281 |
| Single edit: thrA A680* | 461.2140829 | 0.885408333 |
| Single edit: thrA D648* | 371.8330972 | 0.71382062 |
| Single edit: thrA G753* | 266.0877148 | 0.510817619 |
| Single edit: thrA R681* | 180.5327173 | 0.346574786 |
| Single edit: thrA T657* | 89.27068609 | 0.171375967 |
| Single edit: thrA N748* | 64.55536945 | 0.123929134 |
| Single edit: thrA E751* | 55.37857537 | 0.106312131 |
| Single edit: thrA L684* | 2658.955682 | 5.104487491 |
| Single edit: thrA Y594* | 1575.993498 | 3.025488222 |
| Single edit: thrA I685* | 853.7995042 | 1.639067893 |
| Single edit: thrA Y589* | 786.9336594 | 1.510703261 |
| Single edit: thrA A760* | 681.7040503 | 1.308690408 |
| Single edit: thrA A656* | 293.5982728 | 0.563630572 |
| Single edit: thrA A595* | 286.6741841 | 0.550338163 |
| Single edit: thrA G664* | 254.875342 | 0.489292847 |
| Single edit: thrA T462* | 180.2573323 | 0.34604612 |
| Single edit: thrA A504* | 174.7122904 | 0.335401115 |
| Single edit: thrA L647* | 169.2426762 | 0.324900911 |
| Single edit: thrA I749* | 99.18725116 | 0.190413133 |
| Single edit: thrA L705* | 77.78676866 | 0.149329901 |
| Single edit: thrA L509* | 76.79502692 | 0.14742602 |
| Single edit: thrA F709* | 69.92103191 | 0.134229779 |
| Single edit: thrA Y642* | 54.87541602 | 0.105346199 |
| Single edit: thrA A559* | 2136.092315 | 4.100728934 |
| Single edit: thrA L674* | 1550.26902 | 2.976104067 |
| Single edit: thrA K580* | 1236.218222 | 2.373210088 |
| Single edit: thrA I635* | 1047.047284 | 2.010052217 |
| Single edit: thrA L686* | 1028.752267 | 1.974930652 |
| Single edit: thrA A625* | 764.2834504 | 1.467220886 |
| Single edit: thrA I759* | 759.4420248 | 1.457926637 |
| Single edit: thrA A479* | 587.7473447 | 1.128318531 |
| Single edit: thrA R593* | 447.4978962 | 0.859076903 |
| Single edit: thrA S489* | 353.1816389 | 0.678014782 |
| Single edit: thrA D566* | 184.0427987 | 0.353313209 |
| Single edit: thrA R661* | 136.4260058 | 0.261901635 |
| Single edit: thrA S556* | 2189.975432 | 4.204170184 |
| Single edit: thrA G571* | 2097.776237 | 4.027172259 |
| Single edit: thrA D809* | 1863.335412 | 3.577108249 |
| Single edit: thrA V476* | 1722.412304 | 3.306573374 |
| Single edit: thrA V574* | 1392.404904 | 2.673046965 |
| Single edit: thrA L498* | 1105.050946 | 2.121403817 |
| Single edit: thrA Q558* | 1098.067217 | 2.107996913 |
| Single edit: thrA C755* | 1090.565886 | 2.093596354 |
| Single edit: thrA L694* | 1066.027748 | 2.046489659 |
| Single edit: thrA S639* | 1055.546258 | 2.026367986 |
| Single edit: thrA L768* | 995.1955988 | 1.910510777 |
| Single edit: thrA Y588* | 560.4054121 | 1.075829295 |
| Single edit: thrA L547* | 196.996464 | 0.3781808 |
| Single edit: thrA G747* | 159.9947036 | 0.307147264 |
| Single edit: thrA M663* | 118.5982709 | 0.227677127 |
| Single edit: thrA V746* | 98.59469946 | 0.189275592 |
| Single edit: thrA A731* | 72.0219076 | 0.138262901 |
| Single edit: thrA A730* | 53.84365984 | 0.103365502 |
| Single edit: thrA Y564* | 3996.418056 | 7.672059414 |
| Single edit: thrA L811* | 3423.472324 | 6.572156043 |
| Single edit: thrA R812* | 3239.998968 | 6.219936012 |
| Single edit: thrA V754* | 2404.54608 | 4.616088741 |
| Single edit: thrA G634* | 1838.512499 | 3.529454859 |
| Single edit: thrA Q487* | 1602.328936 | 3.076045256 |
| Single edit: thrA L480* | 719.5845179 | 1.381410828 |
| Single edit: thrA S637* | 663.1462888 | 1.273064444 |
| Single edit: thrA A565* | 304.4601917 | 0.584482566 |
| Single edit: thrA V500* | 293.8620349 | 0.564136925 |
| Single edit: thrA K598* | 121.9341716 | 0.234081169 |
| Single edit: thrA K507* | 105.2864095 | 0.202121895 |
| Single edit: thrA N710* | 70.70961053 | 0.13574364 |
| Single edit: thrA L728* | 1655.90606 | 3.178899079 |
| Single edit: thrA F460* | 767.1838152 | 1.472788815 |
| Single edit: thrA S557* | 750.1757878 | 1.440137927 |
| Single edit: thrA V715* | 466.4829595 | 0.895523174 |
| Single edit: thrA A736* | 261.4061471 | 0.501830255 |
| Single edit: thrA F644* | 246.565551 | 0.473340259 |
| Single edit: thrA E662* | 154.0800926 | 0.295792784 |
| Single edit: thrA G740* | 106.7111295 | 0.204856978 |
| Single edit: thrA L725* | 91.07208822 | 0.17483418 |
| Single edit: thrA S723* | 64.4711635 | 0.123767481 |
| Single edit: thrA P668* | 859.8784554 | 1.650737862 |
| Single edit: thrA R692* | 572.1827192 | 1.098438591 |
| Single edit: thrA G610* | 402.2989023 | 0.77230686 |
| Single edit: thrA K631* | 365.0804489 | 0.700857332 |
| Single edit: thrA R601* | 314.6043042 | 0.603956563 |
| Single edit: thrA F572* | 179.7685502 | 0.345107789 |
| Single edit: thrA N721* | 169.0327056 | 0.324497823 |
| Single edit: thrA P531* | 97.48404295 | 0.187143427 |
| Single edit: thrA W521* | 92.76793202 | 0.178089748 |
| Single edit: thrA A711* | 87.70949304 | 0.168378891 |
| Single edit: thrA R737* | 63.86650904 | 0.122606706 |
| Single edit: thrA L540* | 55.96462695 | 0.107437194 |
| Single edit: thrA D463* | 3663.479692 | 7.032906334 |
| Single edit: thrA I466* | 2500.818244 | 4.800905683 |
| Single edit: thrA V742* | 2258.597016 | 4.335905367 |
| Single edit: thrA K682* | 1994.458101 | 3.828828925 |
| Single edit: thrA E467* | 1342.21865 | 2.576702709 |
| Single edit: thrA Y665* | 427.7911234 | 0.821245143 |
| Single edit: thrA T576* | 411.4896155 | 0.789950584 |
| Single edit: thrA T658* | 333.6322596 | 0.640485175 |
| Single edit: thrA G502* | 184.2590358 | 0.353728326 |
| Single edit: thrA L510* | 169.0613669 | 0.324552845 |
| Single edit: thrA L525* | 84.56475151 | 0.162341824 |
| Single edit: thrA D678* | 2016.05926 | 3.870297404 |
| Single edit: thrA E649* | 1457.255214 | 2.797542306 |
| Single edit: thrA K741* | 1216.479897 | 2.335317756 |
| Single edit: thrA A660* | 876.9677646 | 1.683544789 |
| Single edit: thrA L604* | 690.1114546 | 1.324830388 |
| Single edit: thrA D627* | 472.9942153 | 0.908023053 |
| Single edit: thrA N582* | 214.8419124 | 0.412439313 |
| Single edit: thrA I496* | 79.60196339 | 0.152814591 |
| Single edit: thrA H514* | 57.2591372 | 0.10992231 |
| Single edit: thrA H573* | 1981.293303 | 3.803556016 |
| Single edit: thrA A561* | 1750.778761 | 3.361029425 |
| Single edit: thrA H590* | 703.9573771 | 1.351410875 |
| Single edit: thrA D673* | 486.5070663 | 0.933964131 |
| Single edit: thrA N776* | 287.8386872 | 0.5525737 |
| Single edit: thrA E628* | 85.13009237 | 0.163427128 |
| Single edit: thrA G634K | 1003.72 | 4.626532219 |
| Single edit: thrA S637P | 293.12 | 1.351106934 |
| Single edit: thrA S506D | 216.99 | 1.092724406 |
| Single edit: thrA R681H | 265.46 | 0.830782827 |
| Single edit: thrA D673N | 126.37 | 0.395479021 |
| Single edit: thrA L592R | 117.29 | 0.36707111 |
| Single edit: thrA G4775 | 65.43 | 0.32951158 |
| Single edit: thrA A687R | 40.19 | 0.169248991 |
| Single edit: thrA Q84H | 46.2 | 0.144583291 |
| Single edit: thrA D766P | 39.53 | 0.100546303 |
| Single edit: thrA L592G | 21.95 | 0.094255282 |
| Single edit: thrA P767L | 13.03 | 0.033153379 |
| Single edit: thrA Y785H | 8.5 | 0.02161568 |
| Single edit: thrA I616E | 7.94 | 0.020191831 |
| Single edit: thrA A808P | 3.3 | 0.01418256 |
| Single edit: thrA P787W | 2.45 | 0.01052523 |
| Single edit: thrA G676I | 2.13 | 0.009154439 |
| Single edit: thrA G774N | 2.12 | 0.006644687 |
| Single edit: thrA S506I | 2.34 | 0.005942154 |
| Single edit: thrA I616S | 1.57 | 0.00489896 |
| Single edit: thrA N621A | 1.43 | 0.004474036 |

Sequence ID numbers for the single variants are listed in Table 2 below:

TABLE 2
SEQ ID NOs

| SEQ ID NO: | Variant |
|---|---|
| SEQ ID NO: 1* | dapA E84T |
| SEQ ID NO: 2* | dapA J21300 |
| SEQ ID NO: 3* | lysC V339P |
| SEQ ID NO: 4** | garD J23101 |
| SEQ ID NO: 5** | yicL J23100 |
| SEQ ID NO: 6* | lysP R15*** |
| SEQ ID NO: 7** | mgsA J23100 |
| SEQ ID NO: 8* | pckE100Q |
| SEQ ID NO: 9** | amyA J23100 |
| SEQ ID NO: 10* | amyA P15*** |
| SEQ ID NO: 11* | cysN L5*** |
| SEQ ID NO: 12** | dosP J23100 |
| SEQ ID NO: 13** | emrE J23100 |
| SEQ ID NO: 14** | focB J23100 |
| SEQ ID NO: 15** | glnD J23100 |
| SEQ ID NO: 16* | glnE V15*** |
| SEQ ID NO: 17** | hicB J23100 |
| SEQ ID NO: 18** | maeB J23100 |
| SEQ ID NO: 19* | marA Y107D |
| SEQ ID NO: 20* | metL R241E |
| SEQ ID NO: 21* | mfd Y5*** |
| SEQ ID NO: 22* | nupX R5*** |
| SEQ ID NO: 23* | pck H232G |
| SEQ ID NO: 24** | phoB J23100 |
| SEQ ID NO: 25** | purM J23100 |
| SEQ ID NO: 26* | rlmL F5*** |
| SEQ ID NO: 27* | wzxB K5*** |
| SEQ ID NO: 28** | ydgl J23100 |
| SEQ ID NO: 29** | ydjE J23100 |
| SEQ ID NO: 30** | yliE J23100 |
| SEQ ID NO: 31** | yohF J23100 |
| SEQ ID NO: 32* | ytfP N15*** |
| SEQ ID NO: 33* | marA R94*** |
| SEQ ID NO: 34* | marA Y107K |
| SEQ ID NO: 35* | metL P240D |
| SEQ ID NO: 36* | metL V235C |
| SEQ ID NO: 37* | pck G64D |
| SEQ ID NO: 38** | setB J23100 |
| SEQ ID NO: 39** | ydfO J23100 |
| SEQ ID NO: 40** | ydgD J23100 |
| SEQ ID NO: 41** | yejG J23100 |
| SEQ ID NO: 42** | azoR J23100 |
| SEQ ID NO: 43* | cra ESA |
| SEQ ID NO: 44* | cra I6S |
| SEQ ID NO: 45* | cra S18L |
| SEQ ID NO: 46** | dtpA J23100 |
| SEQ ID NO: 47* | fnr R213A |
| SEQ ID NO: 48** | ftsY J23100 |
| SEQ ID NO: 49** | gpp J23100 |
| SEQ ID NO: 50* | lysC V339N |
| SEQ ID NO: 51* | marA K106D |
| SEQ ID NO: 52* | marA Y107M |
| SEQ ID NO: 53* | marA M109Q |
| SEQ ID NO: 54* | metL A50N |
| SEQ ID NO: 55* | metL R207C |
| SEQ ID NO: 56* | metL S230V |
| SEQ ID NO: 57* | metL W2291I |
| SEQ ID NO: 58* | mgsA JS23100 |
| SEQ ID NO: 59* | moaC M15** |
| SEQ ID NO: 60* | oppD Q15** |
| SEQ ID NO: 61* | rob K93P |
| SEQ ID NO: 62** | ycgI J23100 |
| SEQ ID NO: 63** | yeeP J23100 |
| SEQ ID NO: 64** | yihN J23100 |
| SEQ ID NO: 65* | yohO K5*** |
| SEQ ID NO: 66* | metF S15*** |
| SEQ ID NO: 67* | glgB A15*** |
| SEQ ID NO: 68* | yibL E5*** |
| SEQ ID NO: 69* | ppc R880W |
| SEQ ID NO: 70* | metE H641N |
| SEQ ID NO: 71* | metE C726H |
| SEQ ID NO: 72* | rffG V216D |
| SEQ ID NO: 73* | scpB G65N |
| SEQ ID NO: 74* | lysA H373Y |
| SEQ ID NO: 75* | lysA N58I |
| SEQ ID NO: 76* | lysC L377R |
| SEQ ID NO: 77* | tktB E191S |
| SEQ ID NO: 78* | tktB K342S |
| SEQ ID NO: 79* | tktB Q341K |
| SEQ ID NO: 80* | lysC T343I |
| SEQ ID NO: 81* | tktB F283F |
| SEQ ID NO: 82* | lysA M2318K |
| SEQ ID NO: 83* | tktB I102W |
| SEQ ID NO: 84* | dapA G76Y |
| SEQ ID NO: 85* | pck E239Y |
| SEQ ID NO: 86* | pck G208Q |
| SEQ ID NO: 87* | pck K106W |
| SEQ ID NO: 88* | pck S234C |
| SEQ ID NO: 89* | pck T176E |
| SEQ ID NO: 90* | pck N177C |
| SEQ ID NO: 91* | pck T499Y |
| SEQ ID NO: 92* | pck V55Q |
| SEQ ID NO: 93* | dapA L88A |
| SEQ ID NO: 94* | pck V48C |
| SEQ ID NO: 95* | pck V371H |
| SEQ ID NO: 96* | selA I15*** |
| SEQ ID NO: 97* | xy1H T15*** |
| SEQ ID NO: 98* | allC W15*** |
| SEQ ID NO: 99* | metB Q5*** |
| SEQ ID NO: 100* | metB N15*** |
| SEQ ID NO: 101** | metL J23100 |
| SEQ ID NO: 102** | nirB J23100 |
| SEQ ID NO: 103** | yrhC J23100 |
| SEQ ID NO: 104** | prlC J23100 |
| SEQ ID NO: 105** | argK J23100 |
| SEQ ID NO: 106** | glgP J23100 |
| SEQ ID NO: 107** | argA J23100 |
| SEQ ID NO: 108** | tmpR J23112 |
| SEQ ID NO: 109** | ugpQ J23112 |
| SEQ ID NO: 110** | bscF J23112 |
| SEQ ID NO: 111** | lafU J23112 |
| SEQ ID NO: 112** | prfH J23112 |
| SEQ ID NO: 113** | csiR J23106 |
| SEQ ID NO: 114** | feoA J23106 |
| SEQ ID NO: 115** | glgP J23106 |
| SEQ ID NO: 116* | dapA H56M |
| SEQ ID NO: 117* | dapA A79E |
| SEQ ID NO: 118* | dapA H56K |
| SEQ ID NO: 119* | dapA N80H |
| SEQ ID NO: 120* | dapAA79H |
| SEQ ID NO: 121* | dapA A79V |
| SEQ ID NO: 122* | dapA H56T |
| SEQ ID NO: 123* | dapA A79L |
| SEQ ID NO: 124* | dapF L45E |
| SEQ ID NO: 125* | pck M231L |
| SEQ ID NO: 126* | pck G107V |
| SEQ ID NO: 127* | dapA H56E |
| SEQ ID NO: 128* | dapA N80Q |
| SEQ ID NO: 129* | dapA A79N |
| SEQ ID NO: 130* | pck H232L |
| SEQ ID NO: 131* | dapA A79Y |
| SEQ ID NO: 132* | dapA H56Y |
| SEQ ID NO: 133* | dapA L88S |
| SEQ ID NO: 134* | dapA E84N |
| SEQ ID NO: 135* | lysA G148A |
| SEQ ID NO: 136* | dapF N190I |
| SEQ ID NO: 137* | lysC V339A |
| SEQ ID NO: 138** | yfcO J23112 |
| SEQ ID NO: 139* | aroE K15*** |
| SEQ ID NO: 140* | metL A772R |
| SEQ ID NO: 141* | metL D508H |
| SEQ ID NO: 142** | yijF J23101 |
| SEQ ID NO: 143* | dapA A81Q |
| SEQ ID NO: 144* | garD Q274D |
| SEQ ID NO: 145* | dapA L88D |
| SEQ ID NO: 146* | lysC V339D |
| SEQ ID NO: 147* | dapF L46N |
| SEQ ID NO: 148** | ribE J23101 |
| SEQ ID NO: 149* | lysC S338K |
| SEQ ID NO: 150* | argE L5*** |
| SEQ ID NO: 151** | acrD J23106 |
| SEQ ID NO: 152* | dapA L88F |
| SEQ ID NO: 153* | dapA Y106H |
| SEQ ID NO: 154* | yidR V5*** |
| SEQ ID NO: 155* | yicL R5*** |
| SEQ ID NO: 156* | dapA N80A |
| SEQ ID NO: 157* | hupA Q5*** |
| SEQ ID NO: 158* | yhjR G15*** |
| SEQ ID NO: 159* | yhdJ C5*** |
| SEQ ID NO: 160* | adeQ T15*** |
| SEQ ID NO: 161* | dppF A15*** |
| SEQ ID NO: 162* | dapA A81V |
| SEQ ID NO: 163* | dapA A57M |
| SEQ ID NO: 164* | yidG D3*** |
| SEQ ID NO: 165* | pck K106I |
| SEQ ID NO: 166* | metL P766F |
| SEQ ID NO: 167* | thrA G502P |
| SEQ ID NO: 168* | thrA K682G |
| SEQ ID NO: 169* | thrA G634I |
| SEQ ID NO: 170* | thrA Q487D |
| SEQ ID NO: 171* | thrA A808L |
| SEQ ID NO: 172* | thrA V609N |
| SEQ ID NO: 173* | thrA G477E |
| SEQ ID NO: 174* | thrA F603K |
| SEQ ID NO: 175* | thrA L480R |
| SEQ ID NO: 176* | thrA G474L |
| SEQ ID NO: 177* | thrA I685W |
| SEQ ID NO: 178* | thrA S637W |
| SEQ ID NO: 179* | thrA G626E |
| SEQ ID NO: 180* | thrA A777* |
| SEQ ID NO: 181* | thrA G626* |
| SEQ ID NO: 182* | thrA A581* |
| SEQ ID NO: 183* | thrA L683* |
| SEQ ID NO: 184* | thrA M677* |
| SEQ ID NO: 185* | thrA R671* |
| SEQ ID NO: 186* | thrA M586* |
| SEQ ID NO: 187* | thrA R744* |
| SEQ ID NO: 188* | thrA G638* |
| SEQ ID NO: 189* | thrA A680* |
| SEQ ID NO: 190* | thrA D648* |
| SEQ ID NO: 191* | thrA G753* |
| SEQ ID NO: 192* | thrA R681* |
| SEQ ID NO: 193* | thrA T657* |
| SEQ ID NO: 194* | thrA N748* |
| SEQ ID NO: 195* | thrA E751* |
| SEQ ID NO: 196* | thrA L684* |
| SEQ ID NO: 197* | thrA Y594* |
| SEQ ID NO: 198* | thrA I685* |
| SEQ ID NO: 199* | thrA Y589* |
| SEQ ID NO: 200* | thrA A760* |
| SEQ ID NO: 201* | thrA A656* |
| SEQ ID NO: 202* | thrA A595* |
| SEQ ID NO: 203* | thrA G664* |
| SEQ ID NO: 204* | thrA T462* |
| SEQ ID NO: 205* | thrA A504* |
| SEQ ID NO: 206* | thrA L647* |
| SEQ ID NO: 207* | thrA I749* |
| SEQ ID NO: 208* | thrA L705* |
| SEQ ID NO: 209* | thrA L509* |
| SEQ ID NO: 210* | thrA F709* |
| SEQ ID NO: 211* | thrA Y642* |
| SEQ ID NO: 212* | thrA A559* |
| SEQ ID NO: 213* | thrA L674* |
| SEQ ID NO: 214* | thrA K580* |
| SEQ ID NO: 215* | thrA I635* |
| SEQ ID NO: 216* | thrA L686* |
| SEQ ID NO: 217* | thrA A625* |
| SEQ ID NO: 218* | thrA I759* |
| SEQ ID NO: 219* | thrA A479* |
| SEQ ID NO: 220* | thrA R593* |
| SEQ ID NO: 221* | thrA S489* |
| SEQ ID NO: 222* | thrA D566* |
| SEQ ID NO: 223* | thrA R661* |
| SEQ ID NO: 224* | thrA S556* |
| SEQ ID NO: 225* | thrA G571* |
| SEQ ID NO: 226* | thrA D809* |
| SEQ ID NO: 227* | thrA V476* |
| SEQ ID NO: 228* | thrA V574* |
| SEQ ID NO: 229* | thrA L498* |
| SEQ ID NO: 230* | thrA Q558* |
| SEQ ID NO: 231* | thrA C755* |
| SEQ ID NO: 232* | thrA L694* |
| SEQ ID NO: 233* | thrA S639* |
| SEQ ID NO: 234* | thrA L768* |
| SEQ ID NO: 235* | thrA Y588* |
| SEQ ID NO: 236* | thrA L547* |
| SEQ ID NO: 237* | thrA G747* |
| SEQ ID NO: 238* | thrA M663* |
| SEQ ID NO: 239* | thrA V746* |
| SEQ ID NO: 240* | thrA A731* |
| SEQ ID NO: 241* | thrA A730* |
| SEQ ID NO: 242* | thrA Y564* |
| SEQ ID NO: 243* | thrA L811* |
| SEQ ID NO: 244* | thrA R812* |
| SEQ ID NO: 245* | thrA V754* |
| SEQ ID NO: 246* | thrA G634* |
| SEQ ID NO: 247* | thrA Q487* |
| SEQ ID NO: 248* | thrA L480* |
| SEQ ID NO: 249* | thrA S637* |
| SEQ ID NO: 250* | thrA A565* |
| SEQ ID NO: 251* | thrA V500* |
| SEQ ID NO: 252* | thrA K598* |
| SEQ ID NO: 253* | thrA K507* |
| SEQ ID NO: 254* | thrA N710* |
| SEQ ID NO: 255* | thrA L728* |
| SEQ ID NO: 256* | thrA F460* |
| SEQ ID NO: 257* | thrA S557* |
| SEQ ID NO: 258* | thrA V715* |
| SEQ ID NO: 259* | thrA A736* |
| SEQ ID NO: 260* | thrA F644* |
| SEQ ID NO: 261* | thrA E662* |
| SEQ ID NO: 262* | thrA G740* |
| SEQ ID NO: 263* | thrA L725* |
| SEQ ID NO: 264* | thrA S723* |
| SEQ ID NO: 265* | thrA P668* |
| SEQ ID NO: 266* | thrA R692* |
| SEQ ID NO: 267* | thrA G610* |
| SEQ ID NO: 268* | thrA K631* |
| SEQ ID NO: 269* | thrA R601* |
| SEQ ID NO: 270* | thrA F572* |
| SEQ ID NO: 271* | thrA N721* |
| SEQ ID NO: 272* | thrA P531* |
| SEQ ID NO: 273* | thrA W521* |
| SEQ ID NO: 274* | thrA A711* |
| SEQ ID NO: 275* | thrA R737* |
| SEQ ID NO: 276* | thrA L540* |
| SEQ ID NO: 277* | thrA D463* |
| SEQ ID NO: 278* | thrA I466* |
| SEQ ID NO: 279* | thrA V742* |
| SEQ ID NO: 280* | thrA K682* |
| SEQ ID NO: 281* | thrA E467* |
| SEQ ID NO: 282* | thrA Y665* |
| SEQ ID NO: 283* | thrA T576* |
| SEQ ID NO: 284* | thrA T658* |
| SEQ ID NO: 285* | thrA G502* |
| SEQ ID NO: 286* | thrA L510* |
| SEQ ID NO: 287* | thrA L525* |
| SEQ ID NO: 288* | thrA D678* |
| SEQ ID NO: 289* | thrA E649* |
| SEQ ID NO: 290* | thrA K741* |
| SEQ ID NO: 291* | thrA A660* |
| SEQ ID NO: 292* | thrA L604* |
| SEQ ID NO: 293* | thrA D627* |
| SEQ ID NO: 294* | thrA N582* |
| SEQ ID NO: 295* | thrA I496* |
| SEQ ID NO: 296* | thrA H514* |
| SEQ ID NO: 297* | thrA H573* |
| SEQ ID NO: 298* | thrA A561* |
| SEQ ID NO: 299* | thrA H590* |
| SEQ ID NO: 300* | thrA D673* |
| SEQ ID NO: 301* | thrA N776* |
| SEQ ID NO: 302* | thrA E628* |
| SEQ ID NO: 303* | thrA G634K |
| SEQ ID NO: 304* | thrA S637P |
| SEQ ID NO: 305* | thrA S506D |
| SEQ ID NO: 306* | thrA R681H |
| SEQ ID NO: 307* | thrA D673N |
| SEQ ID NO: 308* | thrA L592R |
| SEQ ID NO: 309* | thrA G477S |
| SEQ ID NO: 310* | thrA A687R |
| SEQ ID NO: 311* | thrA Q84H |
| SEQ ID NO: 312* | thrA D766P |
| SEQ ID NO: 313* | thrA L592G |
| SEQ ID NO: 314* | thrA P767L |
| SEQ ID NO: 315* | thrA Y785H |
| SEQ ID NO: 316* | thrA I616E |
| SEQ ID NO: 317* | thrA A808P |
| SEQ ID NO: 318* | thrA P787W |
| SEQ ID NO: 319* | thrA G676I |
| SEQ ID NO: 320* | thrA G774N |
| SEQ ID NO: 321* | thrA S506I |
| SEQ ID NO: 322* | thrA I616S |
| SEQ ID NO: 323* | thrA N621A |
| SEQ ID NO: 324* | pck A532S |

In the table, * after the SEQ ID NO: denotes an amino acid sequence (e.g., a change to the coding region of the protein), ** after the SEQ ID NO: denotes a nucleic acid sequence (e.g., a change to the promoter region or other noncoding region of the protein), “Phenotype FOWT” is fold over wild type (MG1655) in minimal medium; “Phenotype FIOPC” is fold improved over positive control which is MG1655 with E84T single variant. J231XX is a promoter swap at a given locus; *** as part of the variant shorthand denotes hits from the genome-wide knock out library where a triple-stop was inserted at a given position in the locus; * as part of the variant shorthand denotes hits from the genome-wide knock out library where a single-stop was inserted at a given position in the locus.

### EXAMPLES

Mutagenesis libraries specifically targeting the genes the DAP pathway, along with a number of genes whose enzymes convert products feeding into the DAP pathway were designed for saturation mutagenesis. Additionally, to more deeply explore the rest of the E. coli genome for new targets involved in lysine biosynthesis, libraries were designed to target all annotated loci with either premature stop codons (for a knock-out phenotype) or with an insertion of a set of five synthetic promoter variants (for expression modulation phenotypes). Then, the resulting lysine production levels from the single variants were used to conduct additional nucleic acid-guided nuclease editing in two engineered strains of MG1655 to produce double- and triple-variant engineered strains. The first engineered strain is strain MG1655 with a single mutation comprising dapA E84T (SEQ ID NO: 1), the lysine production for which was approximately 500-fold over wildtype lysine production in MG1655. The second engineered strain is strain MG1655 with a double mutation comprising dapA E84T (SEQ ID NO: 1) and dapA J23100 (a mutation in the E. coli dapA promoter, SEQ ID NO. 2), the lysine production for which was approximately 10,000-fold over wildtype lysine production. All libraries were screened at shallow sampling for lysine production via mass spec as described below.

### Editing Cassette and Backbone Amplification and Assembly

Editing Cassette Preparation: 5 nM oligonucleotides synthesized on a chip were amplified using Q5 polymerase in 50 μL volumes. The PCR conditions were 95° C. for 1 minute; 8 rounds of 95° C. for 30 seconds/60° C. for 30 seconds/72° C. for 2.5 minutes; with a final hold at 72° C. for 5 minutes. Following amplification, the PCR products were subjected to SPRI cleanup, where 30 μL SPRI mix was added to the 50 μL PCR reactions and incubated for 2 minutes. The tubes were subjected to a magnetic field for 2 minutes, the liquid was removed, and the beads were washed 2× with 80% ethanol, allowing 1 minute between washes. After the final wash, the beads were allowed to dry for 2 minutes, 50 μL 0.5× TE pH 8.0 was added to the tubes, and the beads were vortexed to mix. The slurry was incubated at room temperature for 2 minutes, then subjected to the magnetic field for 2 minutes. The eluate was removed and the DNA quantified.

Following quantification, a second amplification procedure was carried out using a dilution of the eluate from the SPRI cleanup. PCR was performed under the following conditions: 95° C. for 1 minute; 18 rounds of 95° C. for 30 seconds/72° C. for 2.5 minutes; with a final hold at 72° C. for 5 minutes. Amplicons were checked on a 2% agarose gel and pools with the cleanest output(s) were identified. Amplification products appearing to have heterodimers or chimeras were not used.

Backbone Preparation: A 10-fold serial dilution series of purified backbone was performed, and each of the diluted backbone series was amplified under the following conditions: 95° C. for 1 minute; then 30 rounds of 95° C. for 30 seconds/60° C. for 1.5 minutes/72° C. for 2.5 minutes; with a final hold at 72° C. for 5 minutes. After amplification, the amplified backbone was subjected to SPRI cleanup as described above in relation to the cassettes. The backbone was eluted into 100 μL ddH₂O and quantified before nucleic acid assembly.

Isothermal Nucleic Acid Assembly: 150 ng backbone DNA was combined with 100 ng cassette DNA. An equal volume of 2× Gibson Master Mix was added, and the reaction was incubated for 45 minutes at 50° C. After assembly, the assembled backbone and cassettes were subjected to SPRI cleanup, as described above.

### Transformation of Editing Vector Library into E cloni®

Transformation: 20 μL of the prepared editing vector Gibson Assembly reaction was added to 30 μL chilled water along with 10 μL E cloni® (Lucigen, Middleton, Wis.) supreme competent cells. An aliquot of the transformed cells were spot plated to check the transformation efficiency, where >100× coverage was required to continue. The transformed E cloni® cells were outgrown in 25 mL SOB+100 μg/mL carbenicillin (carb). Glycerol stocks were generated from the saturated culture by adding 500 μL 50% glycerol to 1000 μL saturated overnight culture. The stocks were frozen at −80° C. This step is optional, providing a ready stock of the cloned editing library. Alternatively, Gibson or another assembly of the editing cassettes and the vector backbone can be performed before each editing experiment.

### Creation of New Cell Line Transformed With Engine Vector

Transformation: 1 μL of the engine vector DNA (comprising a coding sequence for MAD7 nuclease under the control of the pL inducible promoter, a chloramphenicol resistance gene, and the μ Red recombineering system) was added to 50 μL EC83 strain E. coli cells. The transformed cells were plated on LB plates with 25 μg/mL chloramphenicol (chlor) and incubated overnight to accumulate clonal isolates. The next day, a colony was picked, grown overnight in LB+25 μg/mL chlor, and glycerol stocks were prepared from the saturated overnight culture by adding 500 μL 50% glycerol to 1000 μL culture. The stocks of EC1 comprising the engine vector were frozen at −80° C.

### Preparation of Competent Cells

A 1 mL aliquot of a freshly-grown overnight culture of EC83 cells transformed with the engine vector was added to a 250 mL flask containing 100 mL LB/SOB+25 μg/mL chlor medium. The cells were grown to 0.4-0.7 OD, and cell growth was halted by transferring the culture to ice for 10 minutes. The cells were pelleted at 8000× g in a JA-18 rotor for 5 minutes, washed 3x with 50 mL ice cold ddH₂O or 10% glycerol, and pelleted at 8000× g in JA-18 rotor for 5 minutes. The washed cells were resuspended in 5 mL ice cold 10% glycerol and aliquoted into 200 μL portions. Optionally at this point the glycerol stocks could be stored at −80° C. for later use.

### Screening of Edited Libraries for Lysine Production

Library stocks were diluted and plated onto 245×245mm LB agar plates (Teknova) containing 100 μg/mL carbenicillin (Teknova) and 25 μg/mL chloramphenicol (Teknova) using sterile glass beads. Libraries were diluted an appropriate amount to yield ˜2000-3000 colonies on the plates. Plates were incubated ˜16 h at 30° C. and then stored at 4° C. until use. Colonies were picked using a QPix™ 420 (Molecular Devices) and deposited into sterile 1.2 mL square 96-well plates (Thomas Scientific) containing 300 μL of overnight growth medium (EZ Rich Defined Medium, w/o lysine (Teknova), 100 μg/mL carbenicillin and 25 μg/mL chloramphenicol). Plates were sealed (AirPore sheets (Qiagen)) and incubated for ˜19 h in a shaker incubator (Climo-Shaker ISF1-X (Kuhner), 30° C., 85% humidity, 250 rpm). Plate cultures were then diluted 20-fold (15 μL culture into 285 μL medium) into new 96-well plates containing lysine production medium (20 g/L ammonium sulfate (Teknova), 200 mM MOPS buffer (Teknova), 3 mg/L Iron(II) sulfate heptahydrate (Sigma), 3 mg/L Manganese (II) sulfate monohydrate (Sigma), 0.5 mg/L Biotin (Sigma), 1 mg/L Thiamine hydrochloride (Sigma), 0.7 g/L Potassium chloride (Teknova), 20 g/L glucose (Teknova), 5 g/L Potassium phosphate monobasic (Sigma), 1 mL/L Trace metal mixture (Teknova), 1 mM Magnesium sulfate (Teknova), 100 μg/mL carbenicillin and 25 μg/mL chloramphenicol). Production plates were incubated for 24 h in a shaker incubator (Climo-Shaker ISF1-X (Kuhner), 30° C., 85% humidity, 250 rpm).

Production plates were centrifuged (Centrifuge 5920R, Eppendorf) at 3,000 g for 10 min to pellet cells. The supernatants from production plates were diluted 100-fold into water (5 μL of supernatant with 495 μL) of water in 1.2 mL square 96-well plates. Samples were thoroughly mixed and then diluted a subsequent 10-fold further into a 50:50 mixture of acetonitrile and water (20 μL sample with 180 μL of the acetonitrile/water mixture) into a 96-well Plate (polypropylene, 335 μL/well, Conical Bottom (Thomas Scientific). Plates were heat sealed and thoroughly mixed.

Lysine concentrations were determined using a RapidFire high-throughput mass spectrometry system (Agilent) coupled to a 6470 Triple Quad mass spectrometer (Agilent). The RapidFire conditions were as follows: Pump 1: 80% acetonitrile (LC/MS grade, Fisher), 20% water (LC/MS grade, Fisher), 1.5 mL/min, Pump 2: 100% water, 1.25 mL/min, Pump 3: 5% acetonitrile, 95% water, 1.25 mL/min. RapidFire method: Aspirate: 600 ms, Load/wash: 2000 ms, Extra wash: 0 ms, Elute: 3000 ms, Re-equilibrate: 500 ms. 10 μL injection loop.

### Mass Spectrometry Conditions For Lysine Detection

Precursor ion: 147.1 m/z, Product ion (quantifying): 84 m/z, Dwell: 20, Fragmentor: 80, Collision energy: 20, Cell accelerator voltage: 4, Polarity: positive Precursor ion: 147.1 m/z, Product ion (qualifying): 130 m/z, Dwell: 20, Fragmentor: 80, Collision energy: 8, Cell accelerator voltage: 4, Polarity: positive Source conditions: Gas Temp: 300° C., Gas Flow: 10 L/min, Nebulizer: 45 psi, Sheath gas temp: 350° C., Sheath gas flow: 11 L/min, Capillary voltage: 3000V (positive), Nozzle voltage: 1500V (positive)

Data was analyzed using MassHunter Quantitative Analysis software (Agilent) with a standard curve of lysine used for quantitation of lysine in the samples. Each 96-well plate of samples contained 4 replicates of the wildtype strain and 4 replicates of the dapA E84T positive control strain to calculate the relative lysine yield of samples compared to the controls. Hits from the primary screen were re-tested in quadruplicate using a similar protocol as described above.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. §112, ¶6.

We claim:

1. An engineered E. coli cell comprising the following variant sequences: a dapA protein having an amino acid sequence of SEQ ID NO: 1 and a dapA gene promoter sequence having a nucleic acid sequence of SEQ ID NO: 2 driving expression of a dapA protein; and comprising additional variant proteins selected from the following variant proteins:
a purM gene promoter sequence having a nucleic acid sequence of SEQ ID NO: 25 driving expression of the purM gene and a dosP gene promoter sequence having a nucleic acid sequence of SEQ ID NO: 12 driving expression of a dosP protein;
the purM gene promoter sequence having the nucleic acid sequence of SEQ ID NO: 25 driving expression of the purM gene and a glnD gene promoter sequence having a nucleic acid sequence of SEQ ID NO: 15 driving expression of a glnD protein;
a pck gene having an amino acid sequence of SEQ ID NO: 324 and the glnD gene promoter sequence having the nucleic acid sequence of SEQ ID NO: 15 driving expression of the glnD protein;
a metL gene having an amino acid sequence of SEQ ID NO: 20;
a glnE gene having an amino acid sequence of SEQ ID NO: 16;
the glnD gene promoter sequence having the nucleic acid sequence of SEQ ID NO: 15 driving expression of the glnD protein; or
a ytfP gene having an amino acid sequence of SEQ ID NO: 32.

2. The engineered E. coli cell of claim 1, comprising the dapA protein having the amino acid sequence of SEQ ID NO: 1; the dapA gene promoter sequence having the nucleic acid sequence of SEQ ID NO: 2 driving expression of the dapA protein; the purM gene promoter sequence having the nucleic acid sequence of SEQ ID NO: 25 driving expression of the purM gene; and the dosP gene promoter sequence having the nucleic acid sequence of SEQ ID NO: 12 driving expression of the dosP protein.

3. The engineered E. coli cell of claim 1, comprising the dapA protein having the amino acid sequence of SEQ ID NO: 1; the dapA gene promoter sequence having the nucleic acid sequence of SEQ ID NO: 2 driving expression of the dapA protein; the purM gene promoter sequence having the nucleic acid sequence of SEQ ID NO: 25 driving expression of the purM gene; and the glnD gene promoter sequence having the nucleic acid sequence of SEQ ID NO: 15 driving expression of the glnD protein.

4. The engineered E. coli cell of claim 1, comprising the dapA protein having the amino acid sequence of SEQ ID NO: 1; the dapA gene promoter sequence having the nucleic acid sequence of SEQ ID NO: 2 driving expression of the dapA protein; the pck gene having the amino acid sequence of SEQ ID NO: 324; and the glnD gene promoter sequence having the nucleic acid sequence of SEQ ID NO: 15 driving expression of the glnD protein.

5. The engineered E. coli cell of claim 1, comprising the dapA protein having the amino acid sequence of SEQ ID NO: 1; the dapA gene promoter sequence having the nucleic acid sequence of SEQ ID NO: 2 driving expression of the dapA protein; and the metL gene having the amino acid sequence of SEQ ID NO: 20.

6. The engineered E. coli cell of claim 1, comprising the dapA protein having the amino acid sequence of SEQ ID NO: 1; the dapA gene promoter sequence having the nucleic acid sequence of SEQ ID NO: 2 driving expression of the dapA protein; and the glnE gene having the amino acid sequence of SEQ ID NO: 16.

7. The engineered E. coli cell of claim 1, comprising the dapA protein having the amino acid sequence of SEQ ID NO: 1; the dapA gene promoter sequence having the nucleic acid sequence of SEQ ID NO: 2 driving expression of the dapA protein; and the glnD gene promoter sequence having the nucleic acid sequence of SEQ ID NO: 15 driving expression of the glnD protein.

8. The engineered E. coli cell of claim 1, comprising the dapA protein having the amino acid sequence of SEQ ID NO: 1; the dapA gene promoter sequence having the nucleic acid sequence of SEQ ID NO: 2 driving expression of the dapA protein; and the ytfP gene having the amino acid sequence of SEQ ID NO: 32.

9. An engineered E. coli cell comprising the following variant sequences: a dapA protein having an amino acid sequence of SEQ ID NO: 1 and comprising an additional variant protein selected from the following variant proteins:
an azoR gene promoter sequence having a nucleic acid sequence of SEQ ID NO: 42 driving expression of an azoR protein;
a cra gene having an amino acid sequence of SEQ ID NO: 43;
a cra gene having an amino acid sequence of SEQ ID NO: 44;
a cra gene having an amino acid sequence of SEQ ID NO: 45;
a dptA gene promoter sequence having a nucleic acid sequence of SEQ ID NO: 46 driving expression of a dptA protein;
an fnr gene having an amino acid sequence of SEQ ID NO: 47;
an ftsY gene promoter sequence having a nucleic acid sequence of SEQ ID NO: 48 driving expression of an ftsY protein;
a glnD gene promoter sequence having a nucleic acid sequence of SEQ ID NO: 15 driving expression of a glnD protein;
a gpp gene promoter sequence having a nucleic acid sequence of SEQ ID NO: 49 driving expression of a gpp protein;
a lysC gene having an amino acid sequence of SEQ ID NO: 50;
a lysC gene having an amino acid sequence of SEQ ID NO: 3; or
a marA gene having an amino acid sequence of SEQ ID NO: 51.

10. The engineered E. coli cell of claim 1, comprising the dapA protein having the amino acid sequence of SEQ ID NO: 1 and the azoR gene promoter sequence having the nucleic acid sequence of SEQ ID NO: 42 driving expression of the azoR protein.

11. The engineered E. coli cell of claim 1, comprising the dapA protein having the amino acid sequence of SEQ ID NO: 1 and the cra gene having the amino acid sequence of SEQ ID NO: 43.

12. The engineered E. coli cell of claim 1, comprising the dapA protein having the amino acid sequence of SEQ ID NO: 1 and the cra gene having the amino acid sequence of SEQ ID NO: 44.

13. The engineered E. coli cell of claim 1, comprising the dapA protein having the amino acid sequence of SEQ ID NO: 1 and the cra gene having the amino acid sequence of SEQ ID NO: 45.

14. The engineered E. coli cell of claim 1, comprising the dapA protein having the amino acid sequence of SEQ ID NO: 1 and the dptA gene promoter sequence having the nucleic acid sequence of SEQ ID NO: 46 driving expression of the dptA protein.

15. The engineered E. coli cell of claim 1, comprising the dapA protein having the amino acid sequence of SEQ ID NO: 1 and the fnr gene having the amino acid sequence of SEQ ID NO: 47.

16. The engineered E. coli cell of claim 1, comprising the dapA protein having the amino acid sequence of SEQ ID NO: 1 and the ftsY gene promoter sequence having the nucleic acid sequence of SEQ ID NO: 48 driving expression of the ftsY protein.

17. The engineered E. coli cell of claim 1, comprising the dapA protein having the amino acid sequence of SEQ ID NO: 1 and the glnD gene promoter sequence having the nucleic acid sequence of SEQ ID NO: 15 driving expression of the glnD protein.

18. The engineered E. coli cell of claim 1, comprising the dapA protein having the amino acid sequence of SEQ ID NO: 1 and the gpp gene promoter sequence having the nucleic acid sequence of SEQ ID NO: 49 driving expression of the gpp protein.

19. The engineered E. coli cell of claim 1, comprising the dapA protein having the amino acid sequence of SEQ ID NO: 1 and the lysC gene having the amino acid sequence of SEQ ID NO: 50.

20. The engineered E. coli cell of claim 1, comprising the dapA protein having an amino acid sequence of SEQ ID NO: 1 and the lysC gene having the amino acid sequence of SEQ ID NO: 3.

21. The engineered E. coli cell of claim 1, comprising the dapA protein having an amino acid sequence of SEQ ID NO: 1 and the marA gene having the amino acid sequence of SEQ ID NO: 51.
